(19) United States
(12) Patent Application Publication
(10) Pub. No.: US 2024/0189158 A1
(43) Pub. Date: Jun. 13, 2024

(54) NONWOVEN AND ABSORBENT ARTICLES HAVING THE SAME

(71) Applicant: The Procter & Gamble Company, Cincinnati, OH (US)

(72) Inventors: Tong TONG, Beijing (CN); Xiaoxin LIU, Beijing (CN); Xiaohui DONG, Beijing (CN); Kun SUN, Guangzhou (CH)

(21) Appl. No.: 18/533,270

(22) Filed: Dec. 8, 2023

(30) Foreign Application Priority Data
Dec. 9, 2022 (WO) PCT/CN2022/137845

(51) Int. Cl. A61F 13/47; A61F 13/511; A61F 13/512

(52) U.S. Cl. CPC A61F 13/47; A61F 13/51121; A61F 13/512; A61F 2013/51026

(57) ABSTRACT

The present disclosure relates to a nonwoven substrate having a top surface and an opposite bottom surface comprising: an upper layer forming the top surface of the nonwoven substrate, the upper layer comprising hydrophobic fibers, and a lower layer forming the bottom surface of the nonwoven substrate, the lower layer comprising hydrophilic fibers, wherein the top surface of the nonwoven substrate has a first contact angle of no lower than about 90 degrees as measured according to Contact Angle Test, wherein the bottom surface of the nonwoven substrate has a second contact angle of lower than about 90 degrees as measured according to Contact Angle Test, wherein the nonwoven has a unitary structure, and wherein the upper layer has a thickness no greater than about 1400 μm as measured according to the Thickness Test.

### CROSS REFERENCE TO RELATED APPLICATION

This application claims foreign priority under 35 U.S.C. § 119 to Chinese Patent Application No. PCT/CN2022/137845, filed on Dec. 9, 2022, which is herein incorporated by reference in its entirety.

### FIELD OF THE INVENTION

The present disclosure relates to nonwoven, and an absorbent article comprising the nonwoven.

### BACKGROUND OF THE INVENTION

Absorbent articles have been used as personal hygiene products, such as sanitary napkins, infant disposable diapers, training pants for toddlers, adult incontinence undergarments, and the like. Such absorbent articles are designed to absorb and contain body exudates, in particularly large quantities of urine, runny BM, and/or menses (together the “fluids”). These absorbent articles may comprise several layers providing different functions, for example, a topsheet, a backsheet, and an absorbent core disposed therebetween, among other layers (e.g., acquisition layer, distribution layer, etc.) as desired.

Nonwovens are widely used as components such as topsheets constituting absorbent articles such as sanitary napkins, infant disposable diapers, personal care disposable diapers, and the like. Various nonwovens have been suggested for use as topsheets for absorbent articles from the standpoints of skin sensation, a feeling of dryness, comfort, absorption of expelled bodily fluids, and/or prevention of fluid flow-back.

One particularly desirable quality of topsheets in absorbent articles is to reduce the amount of time the fluids spend on the topsheets prior to being absorbed by the absorbent article. If the fluids remain on the surfaces of the topsheets for too long of a period of time, wearers may feel unpleasant wetness and discomfort may increase. Another desirable quality of topsheets is prevention of fluid flow-back through a topsheet and provision of dryness feel. Meanwhile, topsheets acquire and retain some fluid in small capillaries that might exist between fibers which may be visually perceptible to the user of the product as an undesirable stain. It is also a desirable characteristic of absorbent articles to present a clean user contacting surface with less stain.

It has been known that a hydrophilic topsheet exhibits a faster acquisition speed compared to a hydrophobic topsheet; however, it tends to cause unpleasant wetness sensation as it traps or retains the fluid, and/or the fluid is flow-back through a topsheet due to high affiliation of constituting fibers to the fluid. Therefore, absorbent articles having hydrophobic topsheets may be preferred by some consumers because they provide dryness feel and good blurring and stain masking benefits regarding menses/urine staining. However, hydrophobic topsheets absorb the fluid only via capillary force which leads to slower acquisition speed and causes fluid leakage problems. Nonwoven topsheets having a hydrophobic upper layer and a hydrophilic bottom layer were suggested for enhancing transfer of the body fluid from the top surface toward the bottom surface of the topsheet and eventually to the absorbent core. WO2018/167883 discloses an absorbent article comprising a laminate nonwoven which comprises a first surface facing the skin of the wearer, the first surface comprising a hydrophobic first layer, and a second surface comprising a hydrophilic lower layer. The first and lower layers are heat fused together at heat-bonded points where the laminate nonwoven has a thickness smaller than that of peripheral portions, and the upper layer has inter-fiber fusion bonded points where constituent fibers of the upper layer are fused together, and have a thickness smaller than that of a peripheral portions. US2016/0067118A discloses an apertured nonwoven laminate for use in an absorbent article, the nonwoven laminate comprising a hydrophobic first nonwoven layer and a hydrophilic second nonwoven layer.

There is a continuous need for a topsheet of absorbent articles providing a rapid fluid acquisition speed at the same time to alleviate back flow of the body fluid and reduce the unpleasant rewet.

There is also a need for a topsheet of absorbent articles providing improved stain masking so that it can provide a clean user contacting surface without compromising fluid handling properties such as a rapid fluid acquisition speed and mitigated rewet.

There is a continuous need for a topsheet of absorbent articles providing smooth and soft sensation without comprising fluid handling properties such as a rapid fluid acquisition speed and mitigated rewet.

### SUMMARY OF THE INVENTION

The present disclosure provides a nonwoven substrate having a top surface and an opposite bottom surface, and comprising an upper layer forming the top surface of the nonwoven substrate and a lower layer forming the bottom surface of the nonwoven substrate, wherein the top surface of the nonwoven substrate has a first contact angle of no lower than about 90 degrees as measured according to Contact Angle Test, wherein the bottom surface of the nonwoven substrate has a second contact angle of lower than about 90 degrees as measured according to Contact Angle Test, and wherein the nonwoven has unitary structure, and wherein the upper layer has a thickness no greater than about 1200 μm as measured according to Thickness Test.

The present disclosure also provides an absorbent article comprising a wearer facing surface, a garment facing surface, a liquid permeable topsheet, a liquid impermeable backsheet, and an absorbent core disposed between the topsheet and the backsheet, wherein the topsheet comprises a nonwoven substrate described herein, the topsheet being disposed in such a way that the upper layer of the nonwoven substrate forms at least part of the wearer facing surface.

These and other features, aspects, and advantages of the present disclosure will become evident to those skilled in the art from a reading of the present disclosure.

### BRIEF DESCRIPTION OF THE DRAWINGS

FIG. 1 is a schematic view of a nonwoven having an upper layer and a lower layer in accordance with the present disclosure.

FIG. 2A is a cross-section image of an exemplary nonwoven of the present disclosure.

FIG. 2B is a cross-section image of another exemplary nonwoven of the present disclosure.

FIG. 3 is a top schematic view of another nonwoven.

FIG. 4 is a top schematic view of an exemplary nonwoven constituting a topsheet.

FIG. 5 is a perspective view of an exemplary absorbent article.

FIG. 6 is a lateral cross-section view along 6-6 of the absorbent article of FIG. 5.

FIG. 7A is an image of an exemplary water droplet having a contact angle greater than 90 degrees according to Contact Angle Test disclosed herein.

FIG. 7B is an image of an exemplary water droplet having a contact angle no greater than 90 degrees according to Contact Angle Test disclosed herein.

FIG. 8A is an image of nonwoven specimen held by an exemplary instrument for measuring nonwoven thickness.

FIG. 8B is an image of a nonwoven specimen to measure nonwoven thickness according to Thickness Test disclosed herein.

FIG. 8C is an image of a nonwoven specimen of FIG. 8B with illustrative denotations to measure nonwoven thickness according to Thickness Test disclosed herein.

FIG. 9A is a perspective view of a strikethrough plate for acquisition time measurement.

FIG. 9B is a plan view of the strikethrough plate of FIG. 9A.

FIG. 9C is a plan view of a 9C-9C direction cross section of the strikethrough plate of FIG. 9B

FIG. 9D is a plan view of part pf the strikethrough plate of FIG. 9B.

FIG. 9E is a plan view of a 9E-9E direction cross section of the strikethrough plate of FIG. 9B.

### DETAILED DESCRIPTION OF THE INVENTION

All ranges are inclusive and combinable. The number of significant digits conveys neither limitations on the indicated amounts nor on the accuracy of the measurements. All numerical amounts are understood to be modified by the word “about” unless otherwise specifically indicated.

The term “absorbent articles”, as used herein, include disposable diapers, sanitary napkins, panty liners, incontinence pads, interlabial pads, breast-milk pads, sweat sheets, animal-use excreta handling articles, animal-use diapers, and the like.

The term “carded” as used herein is used to describe structural features of the fluid management layers described herein. A carded nonwoven utilizes fibers which are cut to a specific length, otherwise known as “staple fibers.” Staple fibers may be any suitable length. For example, staple fibers may have a length of up to 120 mm or may have a length as short as 10 mm. However, if a particular group of fibers are staple fibers, for example viscose fibers, then the length of each of the viscose fibers in the carded nonwoven is predominantly the same, i.e., the staple length. It is worth noting that where additional staple fiber length fiber types are included, for example, polypropylene fibers, the length of each of the polypropylene fibers in the carded nonwoven is also predominantly the same. But the staple length of the viscose and the staple length of the polypropylene may be different.

In contrast, continuous filaments such as by spunbonding or meltblowing processes, do not create staple fibers. Instead, these filaments are of an indeterminate length and are not cut to a specific length as noted regarding their staple fiber length counterparts.

The “longitudinal” direction is a direction running parallel to the maximum linear dimension, typically the longitudinal axis, of the article and includes directions within 45° of the longitudinal direction. “Length” of the article or component thereof, when used herein, generally refers to the size/distance of the maximum linear dimension, or typically to the size/distance of the longitudinal axis, of an article or part thereof.

The “lateral” or “transverse” direction is orthogonal to the longitudinal direction, i.e., in the same plane of the majority of the article and the longitudinal axis, and the transverse direction is parallel to the transverse axis. “Width” of the article or of a component thereof, when used herein, refers to the size/distance of the dimension orthogonal to the longitudinal direction of the article or component thereof, i.e., orthogonal to the length of the article or component thereof, and typically it refers to the distance/size of the dimension parallel of the transverse axis of the article or component.

As used herein, the terms “hydrophilic” and “hydrophobic” have meanings that are well established in the art with respect to the contact angle of water on the surface of a material. Thus, a material having a water contact angle of greater than about 90° as measured by Contact Angle Test is considered hydrophobic, and a material having a water contact angle of less than about 90° as measured by Contact Angle Test is considered hydrophilic.

### Nonwoven Substrate

Nonwoven of the present disclosure comprises a top surface, an opposite bottom surface, an upper layer forming the top surface of the nonwoven substrate, and a lower layer forming the bottom surface of the nonwoven substrate. The upper layer comprises hydrophobic fibers, and the lower layer comprised hydrophilic fibers. The top surface of the nonwoven has a first contact angle of no lower than about 90 degrees as measured according to Contact Angle Test, and the bottom surface of the nonwoven has a second contact angle of lower than about 90 degrees as measured according to Contact Angle Test. The nonwoven has a unitary structure, and the upper layer has a thickness no greater than about 1200 μm as measured according to Thickness Test.

Advantageously, the nonwoven of the present disclosure, when it is used as a topsheet of an absorbent article, provides a fast fluid acquisition speed while maintaining preferable low rewet. The nonwoven of the present disclosure may provide improved stain masking so that it can present a clean user contacting surface. In addition, the nonwoven of the present disclosure may provide a soft and smooth skin feel by employing fine denier fibers at least for the upper layer.

The advantageous properties of the nonwoven of the present invention, without being bound by theory, may be achieved by introducing surface energy difference by the top surface having a first contact angle of no lower than about 90 degrees as measured according to Contact Angle Test, and the bottom surface of the nonwoven having a second contact angle of lower than about 90 degrees as measured according to Contact Angle Test, and by the nonwoven having unitary structure with the upper layer having a thickness no greater than about 1200 μm as measured according to Thickness Test.

Hereinafter, the fiber constituting the nonwoven substrate of the present disclosure, the configurations of the first and the lower layer, and a method for manufacturing the nonwoven, and an absorbent article having the nonwoven substrate are described.

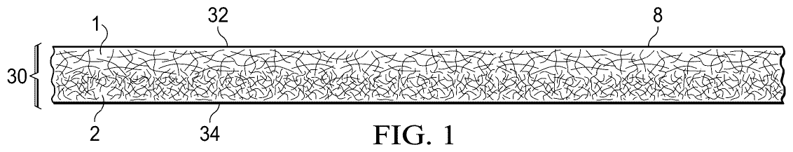

Referring to FIG. 1, when the nonwoven of the present disclosure comprises an upper layer and a lower layer, the upper layer and the lower layer form a unitary structure. A unitary structure herein intends to mean that although it may be formed by several sub-layers that have distinct properties and/or compositions from one another, they are somehow intermixed at the boundary region where the sub-layers are in contact with each other so that, instead of a definite boundary between sub-layers, it would be possible to identify a region where fibers from each sub-layer co-exist, and the different sub-layers transition one into the other. That is, a unitary structure material has a boundary region where fibers from on sub-layer bonds to fibers from an adjacent sub-layer in fiber-to-fiber level. Such a unitary structure is typically built by forming the various sub-layers one on top of the other in a continuous manner, for example using air laid or wet laid deposition. Or each of sub-layer is produced in a separate step and the sub-layers are combined together in a face to face relationship. The sub-layers combined can be integrated via a known integration or bonding process such as spunlacing processes, hydroentangling, through-air bonding and resin bonding. Typically, there is no adhesive used between the sub-layers of the unitary material. However, in some cases, adhesives and/or binders can be present. In comparison, a laminated material has an interface between two adjacent layers where lamination medium such as glue and melting resin, or void space between each layer exists. Such laminate medium and void space negatively impact on fluid penetration and absorbing capability of the laminate material.

When the nonwoven of the present disclosure is described herein, terms of layer (layers), sub-layer(s), and stratum (strata) are used interchangeably.

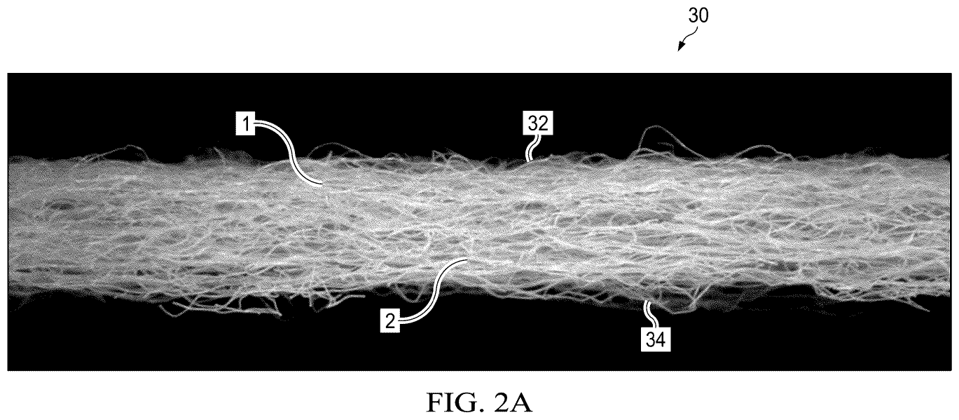

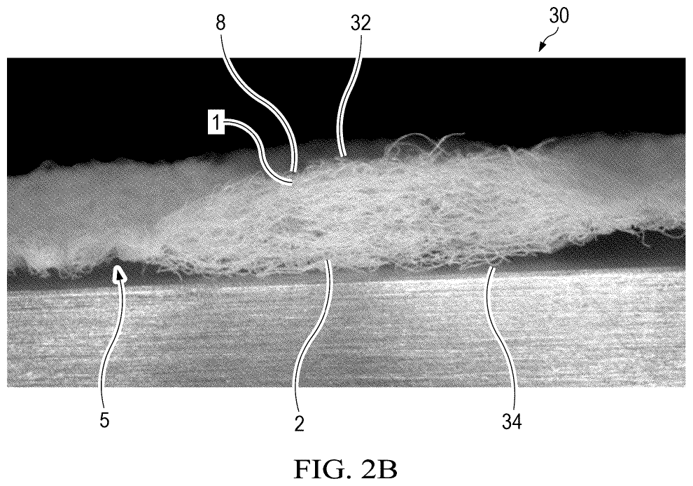

FIG. 2A is a cross section image of an exemplary nonwoven of the present disclosure having an upper layer 1 made by 1.5 denier PE/PET fibers and a lower layer 2 made by 2 denier PE/PET fibers. The nonwoven 30 has unitary structure, and fibers from the upper layer 1 and fibers from the lower layer 2 are intermixed in a boundary region instead of having a definite boundary between the two layers.

Without being bound by theory, the nonwoven of the present disclosure having a unitary structure does not have a lamination interface and may avoid negative impacts on fluid penetration and absorbing capability of the nonwoven. Furthermore, hydrophilic fibers from the lower layer, extends into the upper layer, and the hydrophilic fibers may enhance fluid absorbency from the top surface.

The nonwoven of the present disclosure optionally comprises a plurality of apertures. Referring to FIG. 2B, the nonwoven 30 may comprise a top surface 32, a bottom surface 34, and plurality of apertures 5. Still referring to FIG. 2B, the nonwoven 30 may comprise a plurality of non-aperture area 8 which is a plurality of discrete areas defined by apertures. Each of the plurality of discrete non-aperture areas is defined by a periphery formed by a continuous line of apertures, with at least 80%, or with at least 90% of apertures has an adjacent aperture being spaced apart by an edge-to-edge distance of no more than 3 mm.

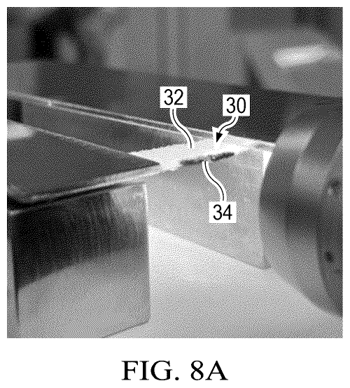

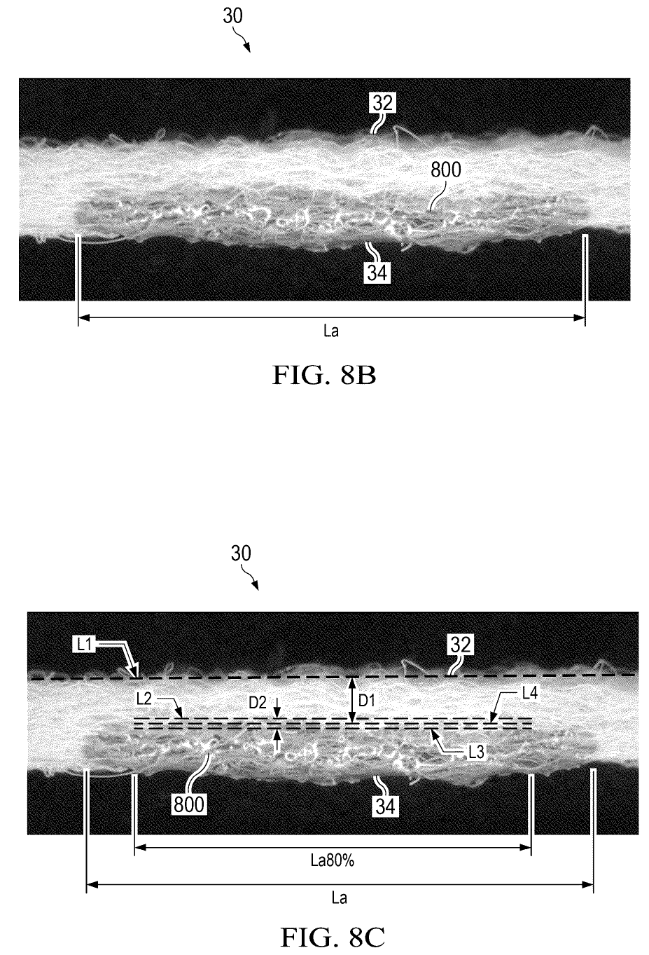

Referring to FIGS. 8B and 8C, nonwoven substrate according to the present disclosure may have a boundary region where hydrophobic fibers from the upper layer and hydrophilic fibers from the lower layer co-exist, the boundary region having a thickness, D2, of about 100 μm, or about 150 μm, or about 170 μm, or about 200 μm in a nonwoven thickness direction as measured according to the Thickness Test.

In one configuration, the hydrophobic fibers constituting the upper layer have a denier no greater than 2.0 denier, or no greater than about 1.5 denier, or no greater than about 1.2 denier, or no greater than about 1.0 denier, or about 0.8 denier. With small denier fibers in the upper layer forming the top surface of the nonwoven, the nonwoven can provide superior softness and smoothness. Though small denier fibers are favorable in the respect with endow the nonwoven with material softness and smoothness, they build more condensed structure with smaller size pours between fibers. Such condensed structure makes the nonwoven more difficult in fluid penetration, especially when the fibers are hydrophobic. In addition, such a condensed structure makes the nonwoven more easily trap fluid within the nonwoven, especially when the fibers are hydrophilic. The nonwoven of the present disclosure can employ small denier fibers such as fibers having a denier no greater than about 1.5 denier, or no greater than about 1.2 denier, or no greater than about 1.0 denier in the upper layer without compensating fluid handling properties. Without being bound by theory, the nonwoven of the present disclosure may have sufficient fluid absorption capability despite the upper layer comprising hydrophobic fibers by having a lower layer comprising hydrophilic layers underneath the upper layer which generates a hydrophilicity gradient to drive the fluid penetration from a top surface of the nonwoven to the bottom, and having a unitary structure where some of hydrophilic fibers from the lower layer extend into the upper layer.

In the embodiment, the hydrophobic fibers constituting the upper layer may have a denier no greater than a denier of the hydrophilic fibers constituting the lower layer.

Capillarity energy is one of common consideration in nonwoven topsheet for better absorbing performance. In order to leverage capillarity energy in the absorbent article design context, a nonwoven topsheet is described to have a capillarity gradient from a top surface to a bottom surface to enlarge capillarity energy. A common nonwoven design to create a capillarity gradient is to make a lower portion of the nonwoven topsheet being more condense, with smaller pore size or more fine denier fibers than an upper portion of the nonwoven topsheet. Meanwhile, it is preferred that an upper portion of nonwoven is formed by fine denier fibers which can endow a soft and smooth touch feel. However, use of fine denier fibers in an upper portion in the topsheet limits choices of fibers for a layer underneath which enables the underneath layer to have more condensed structure. In addition, if a lower portion of a nonwoven topsheet has very condensed structure, it is difficult to find an absorbent material with strong suction capability disposed below the topsheet. Without being bound by theory, nonwoven of the present disclosure can overcome this contradiction by introducing a surface energy gradient, that is by introducing high hydrophilicity gradient from top to bottom, and getting the upper layer and the lower layer fully integrated together. Apertures comprising at least one aperture having at least three adjacent apertures that are spaced apart from by an edge-to-edge space no greater than about 3 mm also play a critical role to overcome the contradiction.

In some configurations, the hydrophobic fibers constituting the upper layer and/or the hydrophilic fibers constituting the lower layer are staple fibers. Staple fibers provide more flexibility in fiber choices than filaments which enable to provide desirable nonwoven properties to meet various different needs.

In some configurations, the nonwoven of the present disclosure is a through-air bonded nonwoven having a unitary structure. The feature that the upper layer and the lower layers are fully and evenly integrated in a fiber level may contribute to the advantageous properties of the nonwoven of the present disclosure. In one configuration, the nonwoven of the present disclosure is a carded air-through nonwoven having a unitary structure. In another configuration, the nonwoven of the present disclosure is a spunbond nonwoven.

A basis weight of the nonwoven of the present disclosure may be appropriately selected depending on the nonwoven application. The nonwoven may have the integral basis weight of the upper layer and the lower layer of the nonwoven from about 10 g/m² to about 100 g/m², or from about 35 g/m² to about 70 g/m². For the use of the nonwoven as a topsheet of an absorbent article, in one embodiment, the integral basis weight of the nonwoven is in the range of from about 30 g/m² to about 70 g/m², or from about 15 g/m² to about 40 g/m², or from about 12 g/m² to about 35 g/m².

In some configurations, the nonwoven comprises an upper layer forming the top surface of the nonwoven and a lower layer forming the bottom surface of the nonwoven. In the configuration, the nonwoven may include at least one intermediate layer between the upper layer and the lower layer.

### Upper Layer

The upper layer of the nonwoven of the present invention comprises hydrophobic fibers. The upper layer may consist essentially of hydrophobic fibers.

The upper layer has a thickness no greater than about 1200 μm, or no greater than about 1050 μm or no greater than about 1100 μm as measured according to the Thickness Test.

Constituting fibers of the upper layer may be natural fibers, synthetic fibers or a combination of natural and synthetic fibers. In one embodiment, the upper layer comprises thermoplastic fibers.

The hydrophobic fibers may be thermoplastic fibers which can selected from the group consisting of polyesters, polypropylenes, polyethylenes, polyethers, polyamides, polyhydroxyalkanoates, polysaccharides, and combinations thereof. Additionally, other synthetic fibers such as rayon, polyethylene, and polypropylene fibers can be used within the scope of the present disclosure. Thermoplastic fibers may be single component fibers (i.e., single synthetic material or a mixture to make up the entire fiber), multicomponent fibers, such as bicomponent fibers (i.e., the fiber is divided into regions, the regions including two or more different synthetic materials or mixtures thereof), and combinations thereof.

Hydrophilic fibers may be rendered hydrophobic by treatment with a hydrophobic treatment such as a hydrophobic surfactant, e.g., by spraying or kiss roll coating hydrophilic fibers with a hydrophobic treatment, by dipping fibers into a hydrophobic treatment or by including a hydrophobic treatment as part of the polymer melt in producing thermoplastic fibers. Upon melting and resolidification, the treatment will tend to remain at the surfaces of the fiber.

The upper layer may comprise semi-synthetic fibers made from polymers, specifically hydroxyl polymers. The topsheet may also comprise semi-synthetic fibers made from polymers, specifically hydroxyl polymers. Non-limiting examples of suitable hydroxyl polymers include polyvinyl alcohol, starch, starch derivatives, chitosan, chitosan derivatives, cellulose derivatives such as viscose, gums, arabinans, galactans, Lyocell (Tencel®), and combinations thereof.

The upper layer may also comprise absorbent fibers. Some examples of absorbent fibers include cotton, pulp, rayon or regenerated cellulose or combinations thereof. The upper layer may also comprise cellulose-based fibers which may be selected from the group consisting of wheat straw fibers, rice straw fibers, flax fibers, bamboo fibers, cotton fibers, jute fibers, hemp fibers, sisal fibers, bagasse fibers, hesperaloe fibers, and combinations thereof.

Several examples of the upper layer may include, but are not limited to: spunbonded nonwovens; carded nonwovens; carded air through nonwovens; spunlace nonwovens, needle punched nonwovens and nonwovens with relatively specific properties to be able to be readily deformed.

The upper layer can be formed from many processes, such as, for example, air laying processes, wetlaid processes, meltblowing processes, spunbonding processes, needle punching processes, and carding processes.

In one form, the upper layer is carded air through nonwoven.

Hydrophobic fibers constituting the upper layer 1 may have a contact angle no lower than about 95 degrees, or no lower than about 100 degrees. The constituent fibers of the upper layer 1 may have a contact angle no greater than 150 degrees, or no greater than 130 degrees, according to Contact Angle Test. The hydrophobicity of the constituent fibers can be adjusted by appropriately adjusting the degree of hydrophobilization treatment of the thermoplastic fibers, for example, the type and content of the hydrophobic treatment.

Hydrophobic fibers constituting the upper layer may have a fiber fineness no greater than 4 denier, or no greater than 2.5 denier, or no greater than 2 denier, or no greater than 1.5 denier, or no greater than 1.2 denier.

The upper layer may have a basis weight from about 5 g/m² to about 20 g/m², or from about from about 8 g/m² to about 15 g/m², about from about 8 g/m² to about 12 g/m², or from about 6 g/m² to about 10 g/m².

### Lower Layer

The lower layer of the nonwoven of the present disclosure comprises hydrophilic fibers. The lower layer may consist essentially of hydrophilic fibers.

Constituting fibers of the lower layer may be natural fibers, synthetic fibers or a combination of natural and synthetic fibers. In one embodiment, the lower layer comprises thermoplastic fibers.

The list of synthetic fibers corresponds to the list disclosed above for the topsheet and the upper layer.

Hydrophobic fibers may be rendered hydrophilic by treatment with a hydrophilic treatment such as a hydrophilic surfactant, e.g., by spraying hydrophobic thermoplastic with a hydrophilic treatment, by dipping the fiber into a treatment or by including a hydrophilic treatment as part of the polymer melt in producing thermoplastic fibers. Upon melting and resolidification, the treatment will tend to remain at the surfaces of the fiber.

Hydrophilic fibers constituting the lower layer may have a fiber fineness no greater than 6 denier, or no greater than 4 denier, or no greater than 2 denier.

Hydrophilic fibers constituting the lower layer may have a contact angle no greater than about 90 degrees, or no greater than about 30 degrees, or no greater than about 10 degrees, or zero degree, according to the Contact Angle Test.

The lower layer may have a basis weight from about 5 to about 70 g/m², or from about 10 to about 60 g/m², or about 10 to about 25 g/m².

All aspects described above for the upper layer, except for the lower layer comprising hydrophilic fibers, are equally applicable to the upper layer in a topsheet comprising upper and lower layers.

### Apertures

The nonwoven of the present disclosure may comprise a plurality of apertures. The apertures may vary in shape. For example, the shape of the apertures as seen from the first surface of the upper layer may be circular, elliptic, rectangular or polygonal. In some configurations, the apertures have a circular shape, an elliptic shape, or a polygonal shape.

The tridimensional shape of the apertures may be cylindrical (e.g., with a circular or elliptic base), prismatic (e.g., with a polygonal base) or truncated cone or pyramidal.

The apertures may be tapered and take a conical shape such that the diameter of the aperture is larger at a part of the aperture proximate to the first surface of the nonwoven than the diameter of the aperture at the bottom edge of the aperture.

Such tapered configuration helps to reduce the risk of rewet, i.e., of body fluids passing back from components underneath the topsheet (such as the absorbent core) into and through the topsheet. For apertured hydrophobic topsheets, rewet occurs predominantly through the apertures. The tapered shape of the apertures can help to reduce rewet, as the diameter of the aperture towards the absorbent core is smaller than the diameter of the aperture in the upper layer.

The plurality of apertures may also vary in width.

The size of apertures may be determined to achieve the desired fluid and/or air penetration performance and other performances expected by wearers. If the apertures are too small, the fluids may not pass through the apertures, either due to poor alignment of the fluid source and the aperture location or due to runny fecal masses, for example, having a diameter greater than the apertures. If the apertures are too large, the area of skin that may be contaminated by “rewet” from the article is increased.

Each of the plurality of apertures may have a size ranging from 0.2 mm² to 1.5 mm², from 0.2 mm² to 1.0 mm², or from 0.25 mm² to 0.5 mm², and/or a diameter ranging from 0.3 mm to 1.5 mm, or from 0.3 mm to 1 mm, or from 0.4 mm to 0.8 mm. The plurality of apertures may have regular shapes selected from the group consisting of circle, oval, triangle, square, rectangle, parallelogram, trapezoid, polygon, hourglass, star, and any combinations thereof.

### Absorbent Article

The nonwoven of the present disclosure exhibits a rapid acquisition of the body fluid and/or maintains dryness of the top surface as it can refrain the body fluid from flowing back to the top surface under pressures.

As such, the nonwoven of the present disclosure can be preferably used in applications in which the nonwoven is in contact with the skin, specifically applications in which the upper layer constitutes the surface that is in contact with the skin in absorbent articles.

Through consumer testing it has been learned that users of feminine hygiene pads most prefer a pad configured with an absorbent structure and a topsheet that exhibits an acquisition time (expressed in seconds) of no greater than 45 s, more preferably no greater than 30 s, and even more preferably no greater that 25 s for the first gush on a fresh pad, when the pad is tested using the Acquisition Time measurement method set forth herein. Acquisition time as measured for purposes herein is a reflection of the absorbent structure/topsheet combination's tendency (or lack thereof) to receive and transfer fluid in a z-direction to the absorbent structure under particular conditions. Rapid acquisition is preferable, but cannot be reduced freely without adversely increasing rewet tendency of the topsheet. It has been also learned that users of feminine hygiene pads most prefer a pad configured with an absorbent structure and a topsheet that exhibits a rewet (expressed gram) of no greater than 0.6 g, more preferably no greater than 0.5 g (accumulated till 9 ml loading). Using the nonwoven topsheet described herein, acquisition time and rewet of the absorbent article can be reduced.

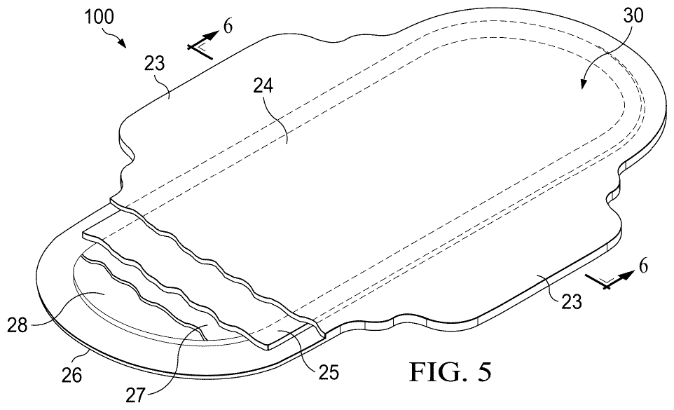

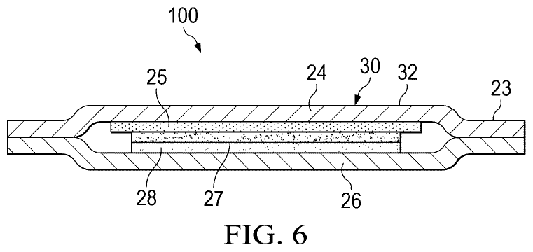

Absorbent articles will now be generally discussed and further illustrated in the form of a sanitary napkin 100 as exemplarily represented in FIG. 5. FIG. 5 is a plan view of the exemplary sanitary napkin 100 in a flattened-out configuration and the garment facing side turned up. FIG. 6 is a lateral cross-section view along 6-6 of the absorbent article of FIG. 5.

Referring to FIGS. 5 and 6, an absorbent article according to the present invention, a sanitary napkin 100 for example, comprises a topsheet 24 comprising nonwoven 30, and has a wearer facing surface and a garment facing surface positioned opposite to the wearer facing surface. The top surface 32 of the nonwoven 30 forms the wearer facing surface of the sanitary napkin 100. The absorbent article further comprises a backsheet 26 having a garment facing surface and a user facing surface positioned oppositely to the garment facing surface, the backsheet 26 being at least partially joined to the topsheet 24. The absorbent article also comprises an absorbent core 28 positioned between the topsheet 24 and the backsheet 26. The absorbent article further comprises a fluid management layer 27 positioned between the topsheet 24 and the absorbent core 28. The absorbent article may further comprise additional acquisition and/or distribution layer (or system) or a secondary topsheet 25, and/or a pair of flaps or wings 23. The topsheet 24, the backsheet 26, the fluid management layer 27, and the absorbent core 28 can be assembled in a variety of well-known configurations.

The backsheet 26 and the topsheet 24 can be secured together in a variety of ways. The topsheet 24 and the backsheet 26 can be joined to each other by using an adhesive, heat bonding, pressure bonding, ultrasonic bonding, dynamic mechanical bonding, or a crimp seal. A fluid impermeable crimp seal can resist lateral migration (“wicking”) of fluid through the edges of the product, inhibiting side soiling of the user's undergarments.

When the absorbent article is a sanitary napkin as shown in FIG. 5, as is typical for sanitary napkins and the like, the sanitary napkin can have panty-fastening adhesive disposed on the garment facing side of backsheet 26. The panty-fastening adhesive can be any of known adhesives used in the art for this purpose, and can be covered prior to use by a release paper, as is well known in the art. If flaps or wings are present, panty fastening adhesive can be applied to the garment facing side so as to contact and adhere to the underside of the user's panties.

An absorbent article according to the present disclosure comprises a topsheet and a liquid impermeable backsheet, and an absorbent core disposed between the topsheet and the backsheet, wherein the topsheet comprises the nonwoven described herein.

The absorbent articles of the present disclosure may be produced industrially by any suitable means. The different layers may thus be assembled using standard means such as embossing, thermal bonding, gluing or any combination thereof.

### Nonwoven Manufacturing Process

The nonwoven according to the present disclosure may be manufactured via various process known in the industry.

The upper layer and the lower layer may be produced separately and bonded or integrated together for example, via thermal and/or glue application. Each of the upper layer and the lower layer may be from a carded web, air-laid web, wet-laid web, and spunbond web, and the like.

The nonwoven according to the present disclosure may be manufactured in a continuous process. For example, the nonwoven is produced by laying a first fibrous web comprising hydrophobic fibers on a conveyor belt, overlaying a second fibrous web comprising hydrophilic fibers on the first fibrous web to obtain a composite fibrous web, and subjecting the composite fibrous web to thermal treatment in order to thermal bond at least a portion of the hydrophobic fibers and hydrophilic fibers. In other examples, the nonwoven is produced using parallel carding machines via a process comprising the steps of forming a first fibrous web comprising hydrophobic fibers, forming a second fibrous web comprising a hydrophilic fibers, forming a composite fibrous web by overlaying the second fibrous web on the first fibrous web; and subjecting the composite fibrous web to thermal treatment in order to thermal bond at least a portion of the hydrophobic fibers and hydrophilic fibers.

The bonding treatment of a composite fibrous web can be conducted using any conventionally known fiber bonding method. Examples of such a bonding method include hot air through-type thermal bonding and ultrasonic bonding.

Aperture of precursor nonwoven can be conducted using any conventionally known nonwoven aperture method. An exemplary aperture equipment is a pair of rolls comprising a pair of rolls.

### Measurement

### 1. Contact Angle Test

All measurements are performed in a laboratory maintained at 23° C.±2° C. and 50%±2% relative humidity.

A rectangular specimen measuring 10 mm×50 mm is cut from a raw material nonwoven or a topsheet of a disposable absorbent article with taking care not to touch the surface of the specimen or not to disturb the structure of the material. The specimen has a length of 5 cm and is aligned with a longitudinal centerline of the absorbent article if it is cut from an absorbent article. The specimen is handled gently by the edges using forceps and mounted flat on a sample stage of an optical microscope such as Keyence VHX 5000 or equivalent. An appropriate light source, magnification and camera position are adjusted to make a cross-section view of the specimen shown clearly.

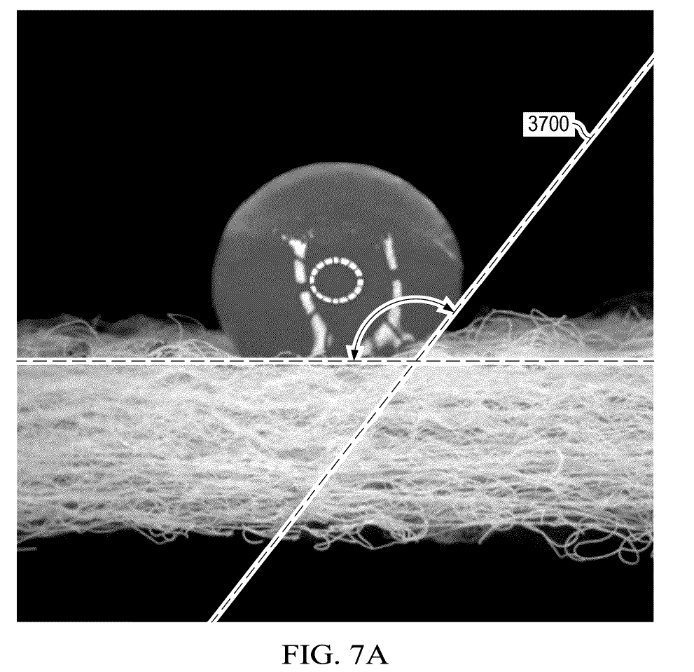

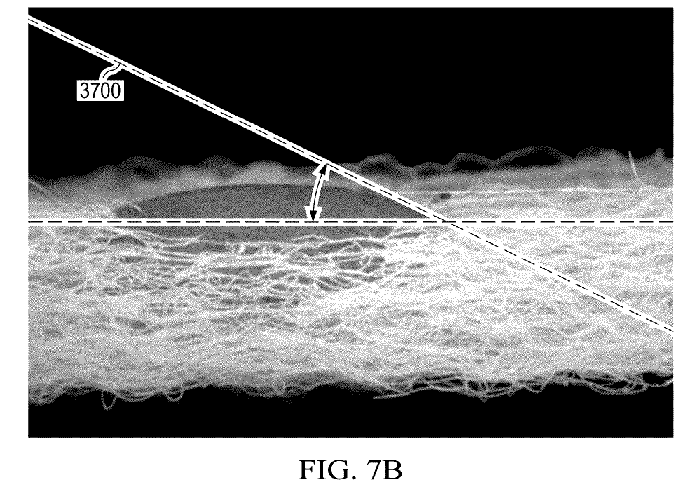

One water droplet with a volume of approximately 0.05 ml is gently deposited onto the specimen from in a close distance no longer than 1 cm above tested surface of the specimen. Keyence VHX 5000 or equivalent instrument is used to obtain a high-resolution image of a water droplet on the tested surface of the nonwoven specimen. These steps are repeated to obtain multiple water droplet images. Suitable water droplet images where each water droplet is oriented such that the projection of the water droplet extending from the nonwoven surface is approximately maximized. The contact angle between the water droplet and the specimen is measured to the nearest 0.1 degree directly from the image taken as is shown via lines 3700 in FIG. 7A and FIG. 7B. FIG. 7A is an exemplary image of a water droplet having a contact angle greater than 90°. FIG. 7B is an exemplary image of a water droplet having a contact angle less than 90°.

The measurement is performed on an area of a test nonwoven where no aperture exists. Five separate droplets are imaged from which ten contact angles, i.e., one on each side of each imaged droplet are measured. The arithmetic mean of the ten contact angle values is calculated to the nearest 0.1 degree and reported as the surface contact angle.

### 2. Nonwoven Thickness Test

When nonwoven is available in a raw material form, a specimen largely smooth with a size of 10 mm×50 mm is cut from the law material nonwoven. When nonwoven is a component of a finished product, a specimen is removed from the component of nonwoven in the finished product using a razor blade to provide a specimen largely smooth with a size of 10 mm×50 mm with care not to touch the surface of the specimen or to disturb the structure of the material. A cryogenic spray such as Sunto™ Freeze Spray, Sunto (HK) International, China may be used to remove the specimen from other components of the finished product.

The specimen is put into an oven with temperature of 80-90° C. for about 30-60 seconds, until the thickness of the specimen has no more increase through longer time.

Referring to FIG. 8A, the specimen is handled gently by the edges using forceps, mounted flat by inserting each lateral side of the specimen into a clamp with the upper layer facing up, and fixed with care not to extend the specimen. The cross section of the specimen is oriented horizontally for the image acquisition under a magnification microscope such as Keyence VHX 5000 or equivalent. The appropriate magnification and light source of the equipment are selected to make the cross section of the specimen shown clearly. Referring to FIG. 8A, one water droplet with color with volume of approximately 0.01 ml is gently disposed with a syringe on a first area on the bottom surface 34 of the specimen. Wait for the water droplet to be sucked into the specimen. Repeat to dispose one water droplet with color with volume of approximately 0.01 ml in an area next to the first area and wait the water droplet is sucked into specimen. Repeat these steps until the length (“La”) of the colored sector 800 sucking the water is at least 10 mm, and take an image of the cross-section view like FIG. 8B.

Referring to FIG. 8C, in the focus view of the specimen 30, draw a line 1 (“L1”) contacting the top surface 32 of the specimen 30. Draw line 2 (“L2”) which is parallel to L1 and contacts the highest point of the colored sector 800 in the center 80% (“La_{80%}”) of the colored sector 800. Draw line 3 (“L3”) which is parallel to L1 and contacts the lowest point of an upper surface of the colored sector 800 in the center 80% of colored sector 800. Draw line 4 (“L4”) which bisects the distance between L2 and L3. The distances D1 between L1 and L4, and D2 between L2 and LA are measured.

In like fashion, a total of three replicate samples are tested for each test nonwoven to be evaluated. The arithmetic mean of D1 of the replicates is calculated to the nearest 1 μm, and reported as the upper layer thickness. The arithmetic mean of D2 of the replicates is calculated to the nearest 1 μm, and reported as the boundary region thickness.

Such method is performed on the non-aperture area of the nonwoven to measure the layer thickness.

### 3. Artificial Menstrual Fluid (“AMF”) Preparation

AMF is composed of a mixture of defibrinated sheep blood, a phosphate buffered saline solution and a mucous component, and has a viscosity between 7.15 cSt to 8.65 cSt at 23±1° C.

Viscosity on the AMF is performed using a low viscosity rotary viscometer such as Cannon LV-2020 Rotary Viscometer with UL adapter (Cannon Instrument Co., State College, US) or equivalent. The appropriate size spindle for the viscosity range is selected, and instrument is operated and calibrated as per the manufacturer. Measurements are taken at 23±1° C. and at 60 rpm. Results are reported to the nearest 0.01 cSt.

### Defibrinated Sheep Blood

Defibrinated sheep blood with a packed cell volume of 38% or greater collected under sterile conditions (available from Cleveland Scientific, Inc., Bath, OH, US) or equivalent is used.

### Phosphate Buffered Saline Solution

The phosphate buffered saline solution consists of two individually prepared solutions (Solution A and Solution B). To prepare 1L of Solution A, add 1.38±0.005 g of sodium phosphate monobasic monohydrate and 8.50±0.005 g of sodium chloride to a 1000 mL volumetric flask and add distilled water to volume. Mix thoroughly. To prepare 1L of Solution B, add 1.42±0.005 g of sodium phosphate dibasic anhydrous and 8.50±0.005 g of sodium chloride to a 1000 mL volumetric flask and add distilled water to volume. Mix thoroughly. Add 450±10 mL of Solution B to a 1000 ml beaker and stir at low speed on a stir plate. Insert a calibrated pH probe (accurate to 0.1) into the beaker of Solution B and add enough Solution A, while stirring, to bring the pH to 7.2±0.1.

### Mucous Component

The mucous component is a mixture of the phosphate buffered saline solution, potassium hydroxide aqueous solution, gastric mucin and lactic acid aqueous solution. The amount of gastric mucin added to the mucous component directly affects the final viscosity of the prepared AMF. A successful range of gastric mucin is usually between 38 to 50 grams. To prepare about 500 mL of the mucous component, add 460±10 mL of the previously prepared phosphate buffered saline solution and 7.5±0.5 mL of the 10% w/v potassium hydroxide aqueous solution to a 1000 mL heavy duty glass beaker. Place this beaker onto a stirring hot plate and while stirring, bring the temperature to 45° C.±5° C. Weigh the pre-determined amount of gastric mucin (±0.50 g) and slowly sprinkle it, without clumping, into the previously prepared liquid that has been brought to 45° C. Cover the beaker and continue mixing. Over a period of 15 minutes bring the temperature of this mixture to above 50° C. but not to exceed 80° C. Continue heating with gentle stirring for 2.5 hours while maintaining this temperature range, then remove the beaker from the hot plate and cool to below 40° C. Next add 1.8±0.2 mL of the 10% v/v lactic acid aqueous solution and mix thoroughly. Autoclave the mucous component mixture at 121° C. for 15 minutes and allow 5 minutes for cool down. Remove the mixture of mucous component from the autoclave and stir until the temperature reaches 23° C.±1° C.

Allow the temperature of the sheep blood and mucous component to come to 23° C.±1° C. Using a 500 mL graduated cylinder, measure the volume of the entire batch of the mucous component and add it to a 1200 ml beaker. Add an equal volume of sheep blood to the beaker and mix thoroughly. Using the viscosity method previously described, ensure the viscosity of the AMF is between 7.15-8.65 cSt. If not, the batch is disposed and another batch is made adjusting the mucous component as appropriate.

The qualified AMF should be refrigerated at 4° C. unless intended for immediate use. AMF may be stored in an air-tight container at 4° C. for up to 48 hours after preparation. Prior to testing, the AMF must be brought to 23° C.±1° C. Any unused portion is discarded after testing is complete.

### 4. Rewet Test

Rewet is measured for an absorbent article loaded with Artificial Menstrual Fluid (“AMF”) as described herein.

The fluid amounts left on a topsheet, i.e., rewet under pressure of 0.1 psi are measured after 3.0 ml, 6.0 ml and 9.0 ml AMF are dispensed. All measurements are performed in a laboratory maintained at 23° C.±2° C. and 50%±2% relative humidity.

Test products are removed from all packaging using care not to press down or pull on the products while handling. No attempt is made to smooth out wrinkles. The test products are conditioned at 23° C.±2° C. and 50%±2% relative humidity for at least 2 hours prior to testing.

Place the test product onto a flat, horizontal surface with the body side facing up and load a strikethrough plate on the center of the test product to apply a pressure of 0.25 psi on the test product.

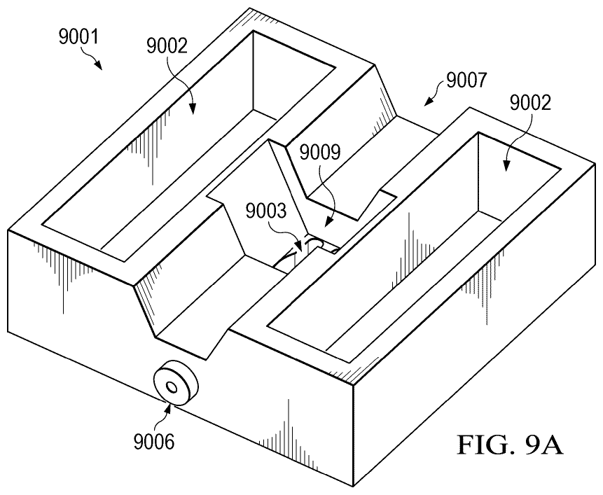

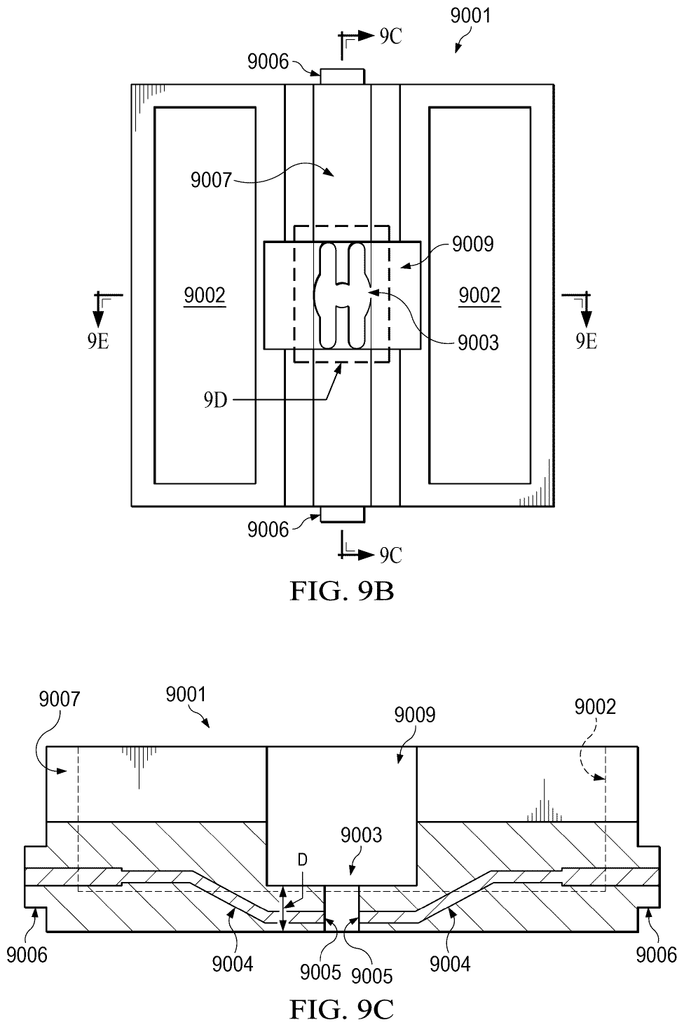

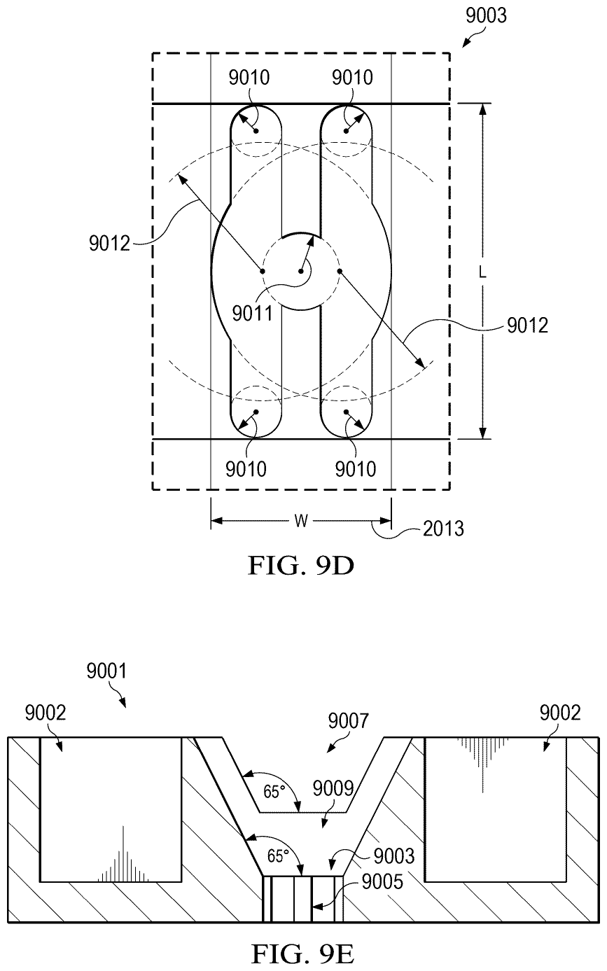

Referring to FIGS. 9A-9E, the strikethrough plate 9001 is constructed of Plexiglas with an overall dimension of 10.2 cm long by 10.2 cm wide by 3.2 cm tall. A longitudinal channel 9007 running the length of the plate is 13 mm deep and 28 mm wide at the top plane of the plate, with lateral walls that slope downward at 65° to a 15 mm wide base. A central test fluid well 9009 is 26 mm long, 24 mm deep and 38 mm wide at the top plane of the plate with lateral walls that slope downward at 65° to a 15 mm wide base. At the base of the test fluid well 9009, there is an “H” shaped test fluid reservoir 9003 open to the bottom of the plate for the fluid to be introduced onto the underlying article. The test fluid reservoir 9003 has an overall length (“L”) of 25 mm, width (“W”) of 15 mm, and depth (“D”) of 8 mm. The longitudinal legs of the reservoir are 4 mm wide and have rounded ends with a radius 9010 of 2 mm. The legs are 3.5 mm apart. The central strut has a radius 9011 of 3 mm and houses the opposing electrodes 9004 6 mm apart. The lateral sides of the reservoir bow outward at a radius 9012 of 14 mm bounded by the overall width, W, of 15 mm. Two wells 9002 (80.5 mm long×24.5 mm wide×25 mm deep) located outboard of the lateral channel, are filled with lead shot to adjust the overall mass of the plate to provide a constraining pressure of 0.25 psi (17.6 gf/cm²) to the test area. Electrodes 9004 are embedded in the plate 9001, connecting the exterior banana jacks 9006 to the inside wall of the fluid reservoir 9003. A circuit interval timer is plugged into the jacks 9006 to the inside wall 9005 of the fluid reservoir 9003.

Use a pipette to carefully dispense 3.0 ml of AMF through the open hole of the strikethrough plate onto the center of the test articles within 2 seconds. Once the gush fluid is acquired, remove the plate and start the timer for 3 minutes. After removing the plate, quicky acquire an image of a topsheet of the test product using a color scanner HP Scanjet G4010 or equivalent, and clean the scanner surface after each scan. The image will be analyzed to measure a stain size on a topsheet under Stain Size Test described below. At the end of 3 minutes, place 5 pieces of filter paper (a typical lab filter paper, for example, Ahlstrom #632 12.7 cm×12.7 cm filter papers) that are pre-weighed (termed as “dry weight”) are placed on top of an approximate center of an area stained with the fluid. Apply the required mass to generate 0.1 psi pressure on the top of the test product, and keep it under pressure for 5 seconds. Weigh the filter papers again (termed as “wet weight”). The difference between the wet weight and dry weight of the filter paper is the light pressure rewet at the added amount of fluid.

Repeat the step above till total 9.0 ml of fluid is dispensed on the test product. Report the rewet values to the nearest 0.001 gram for the gush level of 3.0 ml, 6.0 ml, and 9.0 ml.

In like fashion, a total of three replicate samples are tested for each test product to be evaluated. The arithmetic mean of the replicates is calculated to the nearest 0.001 gram, and reported as the rewet.

### 5. Stain Size Test

The area of a stain visible on a topsheet of an absorbent article due to the fluid left on the topsheet is measured on topsheet images of test products acquired in Rewet Test above for the gush level of 3.0 ml, 6.0 ml and 9.0 ml.

Image analysis is performed using image analysis program such as Image J software (version 1.52p or above, National Institute of Health, USA) or equivalent. The image needs to be distance calibrated with an image of a ruler to give an image resolution, i.e., 7.95 pixels per mm.

Open a topsheet image in Image J. Set the scale according to the image resolution. Crop the image in the center area to make a minimum bounding rectangular selection around the total stain region visible across multiple pad layers. Convert the image type to 8 bit. Apply a Gaussian blur filter to smooth the image by a Gaussian function with a Sigma (radius) of 2. The filtered 8-bit grayscale image is then converted to a binary image using the “Minimum” thresholding method to find the boundary of the stain region on the topsheet (as a result of fluid left on the topsheet) against the lighter-colored stain region from the subsequent layers.

The area of the selected stain region on the topsheet is obtained and recorded as topsheet stain size to the nearest 0.01 cm². This entire procedure is repeated on three substantially similar replicate articles. The arithmetic mean of the three individual recorded measurements is calculated to the nearest 0.01 cm², and reported as the topsheet Stain Size.

### 6. Acquisition Time Test

Acquisition time is measured for an absorbent article loaded with AMF as described herein, using a strikethrough plate and an electronic circuit interval timer. The time required for the absorbent article to acquire a dose of AMF is recorded. All measurements are performed in a laboratory maintained at 23° C.±2° C. and 50%±2% relative humidity.

Referring to FIGS. 9A-9E, the strikethrough plate 9001 is constructed of Plexiglas with an overall dimension of 10.2 cm long by 10.2 cm wide by 3.2 cm tall. A longitudinal channel 9007 running the length of the plate is 13 mm deep and 28 mm wide at the top plane of the plate, with lateral walls that slope downward at 65° to a 15 mm wide base. A central test fluid well 9009 is 26 mm long, 24 mm deep and 38 mm wide at the top plane of the plate with lateral walls that slope downward at 65° to a 15 mm wide base. At the base of the test fluid well 9009, there is an “H” shaped test fluid reservoir 9003 open to the bottom of the plate for the fluid to be introduced onto the underlying article. The test fluid reservoir 9003 has an overall length (“L”) of 25 mm, width (“W”) of 15 mm, and depth (“D”) of 8 mm. The longitudinal legs of the reservoir are 4 mm wide and have rounded ends with a radius 9010 of 2 mm. The legs are 3.5 mm apart. The central strut has a radius 9011 of 3 mm and houses the opposing electrodes 9004 6 mm apart. The lateral sides of the reservoir bow outward at a radius 9012 of 14 mm bounded by the overall width, W, of 15 mm. Two wells 9002 (80.5 mm long×24.5 mm wide×25 mm deep) located outboard of the lateral channel, are filled with lead shot to adjust the overall mass of the plate to provide a constraining pressure of 0.25 psi (17.6 gf/cm²) to the test area. Electrodes 9004 are embedded in the plate 9001, connecting the exterior banana jacks 9006 to the inside wall of the fluid reservoir 9003. A circuit interval timer is plugged into the jacks 9006 to the inside wall 9005 of the fluid reservoir 9003. A circuit interval timer (not shown in the drawings) is plugged into the jacks 9006, and monitors the impedance between the two electrodes 9004, and measures the time from introduction of the AMF into reservoir 9003 until the AMF drains from the reservoir. The timer has a resolution of 0.01 sec.

Test products are removed from all packaging using care not to press down or pull on the products while handling. No attempt is made to smooth out wrinkles. The test samples arc conditioned at 23° C.±2° C. and 50%±2% relative humidity for at least 2 hours prior to testing.

The required mass of the strikethrough plate must be calculated for the specific dimensions of the test article such that a confining pressure of 1.72 kPa is applied. Determine the longitudinal and lateral midpoint of the article's absorbent core. Measure and record the lateral width of the core to the nearest 0.1 cm. The required mass of the strikethrough plate is calculated as the core width multiplied by strikethrough plate length (10.2 cm) multiplied by 17.6 gf/cm² and recorded to the nearest 0.1 g. Add lead shot to the plate to achieve the calculated mass.

Connect the electronic circuit interval timer to the strikethrough plate 9001 and zero the timer. Place the test product onto a flat, horizontal surface with the body side facing up. Gently place the strikethrough plate 9001 onto the center of the test product ensuring that the “H” shaped reservoir 9003 is centered over the test area.

Using a mechanical pipette, accurately pipette 3.00 mL±0.05 mL of AMF into the test fluid reservoir 9003. The fluid is dispensed, without splashing, along the molded lip of the bottom of the reservoir 9003 within a period of 3 seconds or less. After the fluid has been acquired, record the acquisition time to the nearest 0.01 second. Thoroughly clean the electrodes 9004 before each test.

In like fashion, a total of three replicate samples are tested for each test product to be evaluated. The arithmetic mean of the replicates is calculated to the nearest 0.01 sec, and reported as the Acquisition Time (sec).

### EXAMPLES

### Example 1. Nonwoven Preparation

Various nonwoven substrates having configurations as indicated in Table 1 were produced using a parallel carding machine and heat treatment.

Substrate 1: 8 gsm first fibrous web of was fabricated by laying down 1.5 denier hydrophobic PE/PET bicomponent fibers constituting the upper layer on a conveyer belt. 10 gsm second fibrous web was fabricated by laying down 2 denier hydrophilic PE/PP bicomponent fibers constituting the lower layer on a conveyer belt. The second fibrous web was overlaid on the first fibrous web, and the overlaid web was subjected to thermal treatment at the temperatures 130° C.-140° C. The thermal treatment was performed using a hot air through-type thermal treatment apparatus with a breathable conveyor belt. In the heat treatment, the overlaid web was placed on the breathable conveyor belt of the thermal treatment apparatus in such a way that the surface of the first fibrous web was in contact with the breathable conveyor belt.

Substrate 2: Substrate 2 was produced according to the process disclosed with respect to Substrate 1 using 2 denier hydrophobic PE/PET bicomponent fibers for the upper layer and 10 gsm 2 denier hydrophilic PE/PP bicomponent fibers for the lower layer.

Substrate 3: Substrate 3 was produced according to the process disclosed with respect to Substrate 1 using 8 gsm 1.5 denier hydrophobic PE/PET bicomponent fibers for the upper layer and 13 gsm 2 denier hydrophilic PE/PP bicomponent fibers for the lower layer.

Substrate 4: Substrate 4 was produced according to the process disclosed with respect to Substrate 1 using 11 gsm 1.5 denier hydrophobic PE/PET bicomponent fibers for the upper layer and 13 gsm 2 denier hydrophilic PE/PP bicomponent fibers for the lower layer.

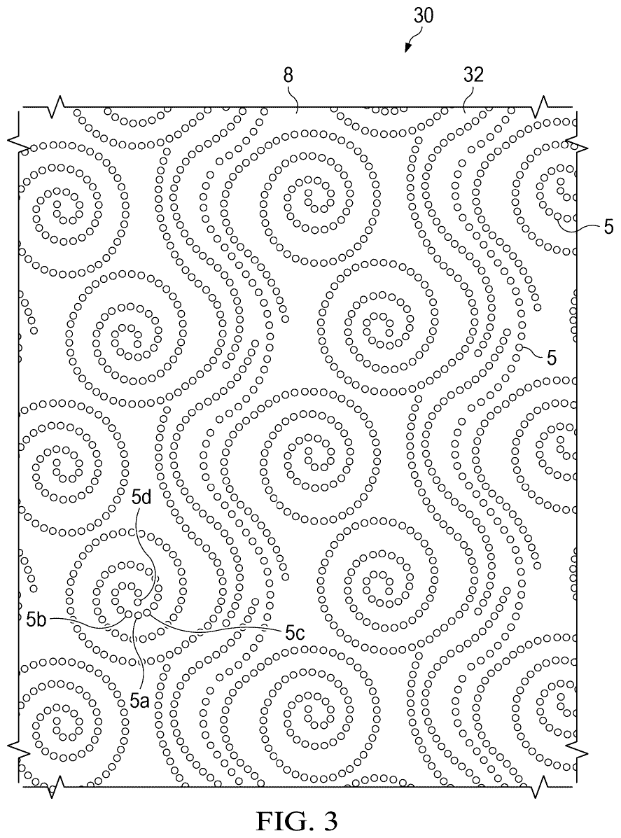

Substrate 5: Substrate 5 was produced using Substrate 4 by forming apertures in a pattern shown in FIG. 3 on Substrate 4.

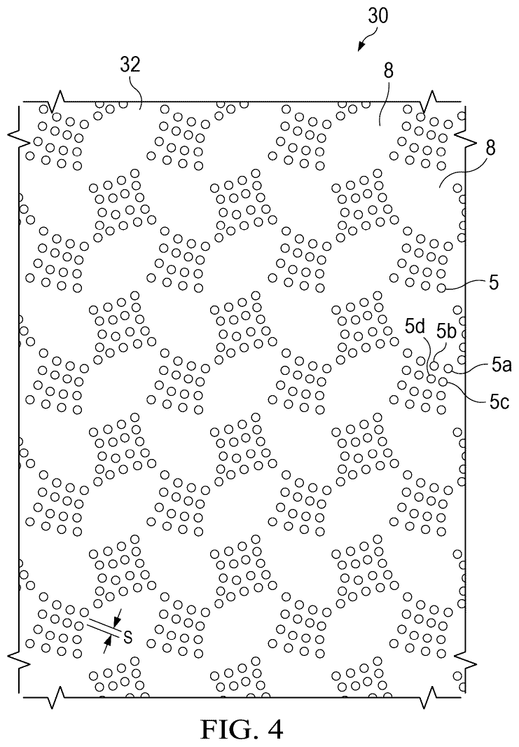

Substrate 6: Substrate 6 was produced using Substrate 4 by forming apertures in a pattern shown in FIG. 4 on Substrate 4.

Substrate 7: Substrate 7 was produced according to the process disclosed with respect to Substrate 1 using 14 gsm 1.5 denier hydrophobic PE/PET bicomponent fibers for the upper layer and 13 gsm 2 denier hydrophilic PE/PP bicomponent fibers for the lower layer.

Substrate 8: Substrate 8 was produced according to the process disclosed with respect to Substrate 1 using 17 gsm 1.5 denier hydrophobic PE/PET bicomponent fibers for the upper layer and 13 gsm 2 denier hydrophilic PE/PP bicomponent fibers for the lower layer.

Substrate 9: Substrate 8 was produced according to the process disclosed with respect to Substrate 1 using 21 gsm 1.5 denier hydrophobic PE/PET bicomponent fibers for the upper layer and 13 gsm 2 denier hydrophilic PE/PP bicomponent fibers for the lower layer.

Substrate 10: 24 gsm of a mixture of 60% 1.5 denier hydrophobic PE/PET bicomponent fibers and 40% 1.5 denier hydrophilic PE/PET bicomponent fibers were laid down on a conveyer belt to obtain a fibrous web. The fibrous web was subjected to thermal treatment at the temperatures 130° C.-140° ° C. to obtain nonwoven. The thermal treatment was performed using a hot air through-type thermal treatment apparatus with a breathable conveyor belt.

Substrate 11: Substrate 11 was produced using Substrate 10 by forming apertures in a pattern shown in FIG. 4 on Substrate 10.

Substrates 1-9 were produced using the same 2D hydrophilic PE/PP and 1.5 D hydrophobic PE/PET. Substrates 10 and 11 were produced using the same 1.5 D hydrophobic PE/PET and 1.5 D hydrophilic PE/PET.

Contact angles on a top surface and an opposite bottom surface in Substrates 1, 3 and 9 were measured according to the Contact Angle Test, and indicated in Table 1 below.

TABLE 1

| | Substrate 1 | Substrate 2 | Substrate 3 | Substrate 4 | Substrate 5 | Substrate 6 |
|---|---|---|---|---|---|---|
| Upper layer | 8 gsm 1.5D | 8 gsm 2D | 8 gsm 1.5D | 11 gsm 1.5D | 11 gsm 1.5D | 11 gsm 1.5D |
| | hydrophobic | hydrophobic | hydrophobic | hydrophobic | hydrophobic | hydrophobic |
| | PE/PET | PE/PET | PE/PET | PE/PET | PE/PET | PE/PET |
| Lower layer | 10 gsm, 2D | 10 gsm 2D | 13 gsm 2D | 13 gsm 2D | 13 gsm 2D | 13 gsm 2D |
| | hydrophilic | hydrophilic | hydrophilic | hydrophilic | hydrophilic | hydrophilic |
| | PE/PP | PE/PP | PE/PP | PE/PP | PE/PP | PE/PP |
| aperture | no aperture | no aperture | no aperture | no aperture | FIG. 3 | FIG. 4 |
| 1^{st} contact | 122.3 | — | 140.8 | — | — | — |
| angle (°) | | | | | | |
| 2^{nd} contact | 0 | — | 28.5 | — | — | — |
| angle (°) | | | | | | |
| Upper layer | | | 828 | 1095 | | — |
| thickness (μm) | | | | | | |
| Boundary region | | | 377 | 269 | | |
| thickness (μm) | | | | | | |

| | Substrate 7 | Substrate 8 | Substrate 9 | Substrate 10 | Substrate 11 |
|---|---|---|---|---|---|
| Upper layer | 14 gsm 1.5D | 17 gsm 1.5D | 21 gsm 1.5D | 24 gsm 60% | 24 gsm 60% |
| | hydrophobic | hydrophobic | hydrophobic | 1.5D hydrophobic | 2D hydrophobic |
| | PE/PET | PE/PET | PE/PET | PE/PET + 40% | PE/PET + 40% |
| | | | | 1.5D hydrophilic | 2D hydrophilic |
| | | | | PE/PET | PE/PET |
| Lower layer | 13 gsm 2D | 13 gsm 2D | 13 gsm 2D | — | — |
| | hydrophilic | hydrophilic | hydrophilic | | |
| | PE/PP | PE/PP | PE/PP | | |
| aperture | no aperture | no aperture | no aperture | no aperture | FIG. 4 |
| 1^{st} contact | — | — | — | 82.8 | — |
| angle (°) | | | | | |
| 2^{nd} contact | — | — | — | 75 | — |
| angle (°) | | | | | |
| Upper layer | 1378 | — | 1800 | | |
| thickness (μm) | | | | | |
| Boundary region | 277 | — | 227 | | |
| thickness (μm) | | | | | |

### Example 2. Absorbent Articles

Sanitary napkins 1-10 as exemplary absorbent articles having topsheets made by nonwoven substrates in Example 1 above were fabricated using a common secondary topsheet, absorbent core and backsheet.

Acquisition speed, and rewet at 0.1 psi/g and 0.5 psi/g of each of the sanitary napkins were tested according to Acquisition Speed Test and Rewet Test disclosed herein. Stain Size and Stain Redness of the sanitary napkins were tested according to Stain Size Test disclosed herein. Table 2 below includes the measurement results.

TABLE 2

| Sanitary napkin | | | 1 | 2 | 3 | 4 | 5 | 6 | 7 | 8 | 9 | 10 |
|---|---|---|---|---|---|---|---|---|---|---|---|---|
| Topsheet | | | Sub- | Sub- | Sub- | Sub- | Sub- | Sub- | Sub- | Sub- | Sub- | Sub- |
| | | | strate 1 | strate 2 | strate 4 | strate 5 | strate 6 | strate 7 | strate 8 | strate 9 | strate 10 | strate 11 |
| Acquisition | 3 | ml | 22.1 | 16.5 | 26.8 | 28.3 | 21.5 | 30.0 | 64.2 | 78.1 | 17.1 | 20.5 |
| time (sec) | 6 | ml | 47.8 | 42.3 | 61.3 | 55.3 | 44.1 | 64.9 | 72.5 | 71.6 | 43.9 | 49.1 |
| | 9 | ml | 61.2 | 53.1 | 79.0 | 75.0 | 56.9 | 86.2 | 94.7 | 93.8 | 49.0 | 61.1 |
| Rewet at | 3 | ml | 0.01 | 0.00 | 0.04 | 0.01 | 0.02 | 0.03 | 0.01 | 0.01 | 0.03 | 0.04 |
| 0.1 psi/g | 6 | ml | 0.04 | 0.01 | 0.13 | 0.06 | 0.11 | 0.12 | 0.05 | 0.06 | 0.15 | 0.15 |
| | 9 | ml | 0.17 | 0.16 | 0.30 | 0.15 | 0.31 | 0.26 | 0.15 | 0.10 | 0.50 | 0.40 |
| | total | | 0.22 | 0.17 | 0.47 | 0.22 | 0.44 | 0.41 | 0.21 | 0.17 | 0.68 | 0.59 |
| Stain size | 3 | ml | 0.27 | 0.87 | 6.80 | 2.70 | 1.52 | 4.95 | 2.80 | 1.93 | 8.34 | 4.77 |
| (cm²) | 6 | ml | 1.45 | 1.86 | 10.19 | 6.21 | 4.86 | 7.19 | 4.15 | 2.78 | 17.11 | 10.00 |
| | 9 | ml | 5.78 | 8.62 | 16.13 | 9.33 | 8.12 | 12.10 | 7.49 | 4.88 | 28.04 | 16.12 |
| Stain | 3 | ml | 8.25 | 11.30 | 15.81 | 10.49 | 8.52 | 12.83 | 10.35 | 8.82 | 16.16 | 12.34 |
| redness | 6 | ml | 9.49 | 12.57 | 16.65 | 12.35 | 11.06 | 14.71 | 12.47 | 11.10 | 20.63 | 13.65 |
| | 9 | ml | 11.54 | 15.74 | 19.29 | 13.86 | 12.28 | 17.31 | 14.40 | 12.55 | 24.26 | 15.47 |

Each of Sanitary napkins 1-6 exhibits an acquisition time of no greater than 45 seconds for the first 3 ml gush on a fresh pad, and a rewet of no greater than 0.5 g (accumulated till 9 ml loading). Sanitary napkins 1 and 2-6 having small denier fibers such as 1.5 denier which can provide desirable superior softness and smoothness still exhibit fast acquisition time and row rewet.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A nonwoven substrate comprising:
a top surface and an opposite bottom surface;
an upper layer forming the top surface of the nonwoven substrate, wherein the upper layer comprises hydrophobic fibers, and
a lower layer forming the bottom surface of the nonwoven substrate, wherein the lower layer comprises hydrophilic fibers,
wherein the top surface of the nonwoven substrate has a first contact angle of no lower than about 90 degrees as measured according to the Contact Angle Test,
wherein the bottom surface of the nonwoven substrate has a second contact angle of lower than about 90 degrees as measured according to the Contact Angle Test,
wherein the nonwoven substrate has unitary structure, and
wherein the upper layer has a thickness no greater than about 1400 μm as measured according to the Thickness Test.

2. The nonwoven substrate according to claim 1, wherein the hydrophobic fibers and the hydrophilic fibers have different linear densities.

3. The nonwoven substrate according to claim 1, wherein the hydrophobic fibers and the hydrophilic fibers have the same linear density.

4. The nonwoven substrate according to claim 1, wherein the upper layer consists essentially of hydrophobic fibers.

5. The nonwoven substrate according to claim 4, wherein the hydrophobic fibers have a linear density no greater than 2.0 denier.

6. The nonwoven substrate according to claim 4, wherein the hydrophobic fibers have a linear density no greater than 1.5 denier.

7. The nonwoven substrate according to claim 1, wherein the hydrophobic fibers are staple fibers.

8. The nonwoven substrate according to claim 1, wherein the hydrophilic fibers are staple fibers.

9. The nonwoven substrate according to claim 1, wherein the lower layer consists essentially of hydrophilic fibers.

10. The nonwoven substrate according to claim 1, wherein the nonwoven substrate does not comprise an aperture.

11. The nonwoven substrate according to claim 1, wherein the nonwoven substrate does not comprise embossing bonding points.

12. The nonwoven substrate according claim 1, wherein the nonwoven substrate comprises a boundary region wherein the hydrophobic fibers from the upper layer and the hydrophilic fibers from the lower layer co-exist, the boundary region having a thickness of at least about 100 μm.

13. The nonwoven substrate according to claim 1, wherein the nonwoven is a carded air-through nonwoven.

14. The nonwoven substrate according to claim 1, wherein the difference between the first contact angle and the second angle is at least about 10 degrees.

15. The nonwoven substrate according to claim 1, wherein the nonwoven substrate further comprises a plurality of apertures.

16. An absorbent article comprising:
a wearer facing surface;
a garment-facing surface;
a liquid permeable topsheet;
a liquid impermeable backsheet; and
an absorbent core disposed between the topsheet and the backsheet;
wherein the topsheet comprises a nonwoven substrate, the nonwoven substrate comprising:
a top surface and an opposite bottom surface;
an upper layer forming the top surface of the nonwoven substrate, wherein the upper layer comprises hydrophobic fibers, and
a lower layer forming the bottom surface of the nonwoven substrate, wherein the lower layer comprises hydrophilic fibers,
wherein the top surface of the nonwoven substrate has a first contact angle of no lower than about 90 degrees as measured according to the Contact Angle Test,
wherein the bottom surface of the nonwoven substrate has a second contact angle of lower than about 90 degrees as measured according to the Contact Angle Test,
wherein the nonwoven substrate has unitary structure, and
wherein the upper layer has a thickness no greater than about 1400 μm as measured according to the Thickness Test;
wherein the top surface of the nonwoven substrate is the wearer facing surface of the absorbent article.

17. The absorbent article of claim 16 further comprising a fluid distribution layer between the topsheet and the absorbent core.

18. The absorbent article of claim 16, wherein the absorbent article is a sanitary napkin.

19. The nonwoven substrate according to claim 16, wherein the upper layer consists essentially of hydrophobic fibers.

20. The nonwoven substrate according to claim 19, wherein the hydrophobic fibers have a linear density no greater than 2.0 denier.
